(19) United States
(12) Patent Application Publication
(10) Pub. No.: US 2025/0196126 A1
(43) Pub. Date: Jun. 19, 2025

(54) ENVIRONMENTAL DEOXYRIBONUCLEIC ACID (EDNA) SURVEILLANCE UNMANNED AERIAL VEHICLE (UAV)

(71) Applicant: International Business Machines Corporation, Armonk, NY (US)

(72) Inventors: Heather Nicole Polgrean, Lagrangeville, NY (US); Evan Armon Imbriglio, Newburgh, NY (US); Ryan Seifts, POUGHKEEPSIE, NY (US); Nnaemeka Mcdonald Eluagu, Poughkeepsie, NY (US)

(21) Appl. No.: 18/540,533

(22) Filed: Dec. 14, 2023

(51) Int. Cl. B01L 3/00; A61L 2/20; A61L 101/02; C12Q 1/6806; C12Q 1/686; C12Q 1/6869

(52) U.S. Cl. CPC B01L 3/50; A61L 2/202; C12Q 1/6806; C12Q 1/686; C12Q 1/6869; A61L 2101/02; B01L 2200/141; B01L 2300/0663; B01L 2300/0864

(57) ABSTRACT

An unmanned aerial vehicle (UAV) for environmental surveillance. A sample capture chamber is configured to collect a sample from a given location. An environmental deoxyribonucleic acid (eDNA) analyzer is configured to extract eDNA from the collected sample and to perform a quantitative polymerase chain reaction (qPCR) analysis on the extracted eDNA. A UAV processor is configured to record results of the analysis, the results including an identification of the given location.

### BACKGROUND

The present invention relates generally to the electrical, electronic and computer arts and, more particularly, to unmanned aerial vehicles.

Different organisms, and even different life stages of a given organism, shed Deoxyribonucleic Acid (DNA) into the environment at various rates. The rate of DNA degradation is highly dependent on environmental variables, such as temperature and microbial activity, making it difficult to accurately estimate species density and how recently a species was present in a given environment. Diligent investigation of environment-specific factors that influence the degradation rate, and lab-based studies to quantify rates of DNA shedding, can overcome these challenges.

Surveilling an environment for pathogens and other environmental conditions can require time and resources that may stress the available resources. For example, surveilling for a pathogen in a city environment may require experts to collect samples and transport them to a lab for analysis. This can be problematic, as procurement and analysis of the results for mitigating health dangers may be time-critical and since the collected samples may degrade over time, making the eventual analysis suspect. While aquatic drones have been utilized for eDNA collection in aquatic environments, they typically are configured without global positioning system (GPS) data caching and are unsuitable for collecting samples in non-aquatic environments. Aquatic drones capture environmental samples and generally return them to a secondary testing location where they can be analyzed.

Aerial drones have been utilized for environmental sampling. Aerial drones store samples and generally return to a secondary location where the samples are collected from the drone.

### BRIEF SUMMARY

Principles of the invention provide systems and techniques for an environmental deoxyribonucleic acid surveillance unmanned aerial vehicle. In one aspect, an exemplary method includes the operations of collecting an eDNA sample on an unmanned aerial vehicle; extracting deoxyribonucleic acid (DNA) from the collected eDNA sample on the unmanned aerial vehicle; performing an analysis on the extracted eDNA on the unmanned aerial vehicle; and transmitting a result of the analysis.

In one aspect, an unmanned aerial vehicle includes a sample capture chamber configured to collect a sample from a given location; an eDNA analyzer configured to extract eDNA from the collected sample and to perform a quantitative polymerase chain reaction (qPCR) analysis on the extracted eDNA; and a UAV processor configured to record results of the analysis, the results including an identification of the given location.

In one aspect, a computer program product includes one or more tangible computer-readable storage media and program instructions stored on at least one of the one or more tangible computer-readable storage media, the program instructions executable by a processor, the program instructions including collecting an eDNA sample on an unmanned aerial vehicle; extracting DNA from the collected eDNA sample on the unmanned aerial vehicle; performing an analysis on the extracted eDNA on the unmanned aerial vehicle; and transmitting a result of the analysis.

Techniques as disclosed herein can provide substantial beneficial technical effects, as will be discussed further below. Features and advantages will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIG. 1A is a high-level block diagram of an eDNA UAV configured to perform remote surveillance in an aerial environment, in accordance with an example embodiment;

FIG. 1B is a high-level block diagram of an eDNA UAV configured to perform remote surveillance of an environment, in accordance with an example embodiment;

FIG. 1C is a block diagram of an example sample capture chamber that includes a plurality of sub-chambers, in accordance with an example embodiment;

FIG. 2 is a flowchart for controlling an eDNA UAV configured to perform remote surveillance of an environment, in accordance with an example embodiment; and

FIG. 3 depicts a computing environment according to an embodiment of the present invention.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

Principles of inventions described herein will be in the context of illustrative embodiments. Moreover, it will become apparent to those skilled in the art given the teachings herein that numerous modifications can be made to the embodiments shown that are within the scope of the claims. That is, no limitations with respect to the embodiments shown and described herein are intended or should be inferred.

Given the discussion herein (reference characters refer to the drawings discussed below), it will be appreciated that, in general terms, an exemplary method, according to an aspect of the invention, includes the operations of collecting an eDNA sample on an unmanned aerial vehicle 210 (operation 262); extracting DNA from the collected eDNA sample on the unmanned aerial vehicle 210 (operation 266); performing an analysis on the extracted eDNA on the unmanned aerial vehicle 210 (operation 270); and transmitting a result of the analysis (operation 274). These features enable a surveillance UAV to collect and analyze eDNA on-board; enable the unmanned aerial vehicle 210 to detect and evaluate eDNA remotely via a UAV for the purpose of sampling extant populations, monitoring environmental biodiversity and environmental health, facilitating conservational efforts and the like without the need to tranquilize and harvest samples from living organisms; enable the unmanned aerial vehicle 210 to facilitate the study and monitoring of species that are invasive, elusive or endangered without introducing anthropogenic stressors due to human interactions; enable the unmanned aerial vehicle 210 to perform intelligent tracking of eDNA in an aerial environment; enable the unmanned aerial vehicle 210 to perform fast identification of pathogens in an aerial environment; enable the unmanned aerial vehicle 210 to detect the presence of pests in and around crops, soil, water, air, and the like; enable the unmanned aerial vehicle 210 to detect harmful pathogens (such as measles) in urban areas; and enable the unmanned aerial vehicle 210 to monitor uninhabited or hard to reach locations that would otherwise require intensive human effort. The unmanned aerial vehicle can be of a helicopter or fixed wing type and can include, in the case of a helicopter type, a fuselage with motors and rotors attached, or in the case of a fixed wing type, a fuselage with attached wings, empennage, motor, and propellor. The fuselage can hold the components of one or more embodiments of the invention. The skilled artisan, given the teachings herein, can adapt known helicopter or fixed wing drones to implement aspects of the invention.

In one example embodiment, a sample capture chamber 216 is sanitized, using ozone, following the collection of each eDNA sample. This feature provides the technical effect of enabling the unmanned aerial vehicle 210 to collect and process a plurality of eDNA samples without landing the unmanned aerial vehicle 210 to sanitize the sample capture chamber 216 between collections.

In one example embodiment, the extracted DNA is purified on the unmanned aerial vehicle 210. This feature provides the technical effect of enabling the unmanned aerial vehicle 210 to process a contaminated eDNA sample in-flight.

In one example embodiment, the unmanned aerial vehicle (UAV) 210 flies to a target location (operation 254). This feature provides the technical effect of enabling the unmanned aerial vehicle 210 to expand the eDNA sampling area.

In one example embodiment, an image from the unmanned aerial vehicle 210 is captured, image processing is performed on the captured image and the target location is identified based on the image processing (operation 254). This feature provides the technical effect of enabling the unmanned aerial vehicle 210 to autonomously locate a geographical area of interest for collecting eDNA samples.

In one example embodiment, the performance of the image processing and the identifying the target subject based on the image processing is repeated (operation 258). This feature provides the technical effect of enabling the unmanned aerial vehicle 210 to autonomously locate a plurality of geographical areas of interest for collecting eDNA samples.

In one example embodiment, the collected eDNA sample is tagged with one or more of a collection location and a time of collection. This feature provides the technical effect of enabling the unmanned aerial vehicle 210 to tag collected eDNA samples for further analysis at a land-based laboratory.

In one aspect, an unmanned aerial vehicle 210 includes a sample capture chamber 216 configured to collect a sample from a given location; an eDNA analyzer 224 configured to extract eDNA from the collected sample and to perform a quantitative polymerase chain reaction (qPCR) analysis on the extracted eDNA; and a UAV processor 212 configured to record results of the analysis, the results including an identification of the given location. The eDNA analyzer 224 may be split into two devices, one device configured to extract eDNA from the collected sample and one device configured to perform a quantitative polymerase chain reaction (qPCR) analysis on the extracted eDNA.

In one example embodiment, a camera and image processing unit 232 is coupled to the UAV processor 212 and is configured to capture and process an image to identify the given location. This feature provides the technical effect of enabling the unmanned aerial vehicle 210 to autonomously locate a geographical area of interest for collecting eDNA samples.

In one example embodiment, the sample capture chamber 216 further includes an air intake valve 240 configured to capture the sample from an atmosphere. This feature provides the technical effect of enabling the unmanned aerial vehicle 210 to capture an eDNA sample that is suspended in air.

In one example embodiment, the sample capture chamber 216 further includes a purifying mechanism configured to pump ozone into the sample capture chamber 216 to sanitize the sample capture chamber 216. This feature provides the technical effect of enabling the unmanned aerial vehicle 210 to collect and process a plurality of eDNA samples without landing the unmanned aerial vehicle 210 to sanitize the sample capture chamber 216.

In one example embodiment, the sample capture chamber 216 further includes a plurality of sub-chambers 242-1, 242-2 configured to collect and analyze the collected sample and one or more additional samples in a pipelined manner. This feature provides the technical effect of enabling the unmanned aerial vehicle 210 to collect a plurality of eDNA samples for further analysis at a land-based laboratory without landing the unmanned aerial vehicle 210 between different collections. This feature also provides the technical effect of enabling the unmanned aerial vehicle 210 to collect one eDNA sample while simultaneously analyzing another eDNA sample. In one example embodiment, the eDNA analyzer 224 further includes one or more enzyme containers, each enzyme container 246 configured to hold a corresponding enzyme for replicating specific fragments of a given target species. This feature also provides the technical effect of enabling the unmanned aerial vehicle 210 to analyze certain types of eDNA samples while in-flight.

In one example embodiment, the eDNA analyzer 224 is further configured to detect a target DNA sequence based on a result of the quantitative polymerase chain reaction (qPCR) analysis. This feature also provides the technical effect of enabling the unmanned aerial vehicle 210 to use the quantitative polymerase chain reaction (qPCR) analysis to detect a particular DNA sequence while in-flight.

In one example embodiment, a sample storage chamber 220 is configured to store the sample in the sample capture chamber 216 for future analysis. This feature provides the technical effect of enabling the unmanned aerial vehicle 210 to collect an eDNA sample for further analysis at a land-based laboratory.

In one example embodiment, a sensor unit 228 is coupled to the UAV processor 212 and is configured to determine a current location of the apparatus. This feature provides the technical effect of enabling the unmanned aerial vehicle 210 to tag a collected eDNA sample with the location of the collection and to maneuver the unmanned aerial vehicle 210 to a target location for collecting eDNA samples.

In one aspect, a computer program product includes one or more tangible computer-readable storage media and program instructions stored on at least one of the one or more tangible computer-readable storage media, the program instructions executable by a processor, the program instructions including collecting an eDNA sample on an unmanned aerial vehicle 210 (operation 262); extracting DNA from the collected eDNA sample on the unmanned aerial vehicle 210 (operation 266); performing an analysis on the extracted eDNA on the unmanned aerial vehicle 210 (operation 270); and transmitting a result of the analysis (operation 274).

As used herein, “facilitating” an action includes performing the action, making the action easier, helping to carry the action out, or causing the action to be performed. Thus, by way of example and not limitation, instructions executing on a processor might facilitate an action carried out by instructions executing on a remote processor, by sending appropriate data or commands to cause or aid the action to be performed. Where an actor facilitates an action by other than performing the action, the action is nevertheless performed by some entity or combination of entities.

Techniques as disclosed herein can provide substantial beneficial technical effects. Some embodiments may not have these potential advantages and these potential advantages are not necessarily required of all embodiments. By way of example only and without limitation, one or more embodiments may provide one or more of:
a surveillance UAV configured to collect and analyze eDNA on-board;
techniques to detect and evaluate eDNA remotely via a UAV for the purpose of sampling extant populations, monitoring environmental biodiversity and environmental health, facilitating conservational efforts and the like without the need to tranquilize and harvest samples from living organisms;
an eDNA UAV that facilitates the study and monitoring of species that are invasive, elusive or endangered without introducing anthropogenic stressors due to human interactions;
intelligent identification and tracking of environmental areas likely to harbor target eDNA;
intelligent tracking of eDNA in an aerial environment;
a remote quantitative polymerase chain reaction (qPCR) analyzer that enables the remote analysis of environmental samples in a timely manner;
fast identification of pathogens in an aerial environment;
utilization of eDNA eliminates the need for observation and/or the capturing of species of interest, and eliminates the need for extensive documentation or human effort;
early detection of invasive species and endangered species monitoring;
detection of the presence of pests in and around crops, soil, water, air, and the like;
detection of harmful pathogens in urban areas (such as measles);
enables a determination of a species prevalence in an environment and population statistics without needing to count or capture individuals;
enables the study of public health via pathogen detection;
reduced resources for identifying and tracking eDNA;
increased ability to monitor uninhabited or hard to reach locations that would otherwise require intensive human effort;
ability to process and/or analyze samples in the field; and
automated surveillance of specified or high-risk locations (such as farms, reservoirs, cities, and the like).

### Environmental DNA

Environmental DNA (eDNA) is DNA that can be collected from a variety of environmental samples including soil, fresh water, salt water, snow, air, and the like, rather than from an organism directly. Example sources of eDNA might include oil shed from a bird's feathers that lingers in the air, mucus, gametes, shed skin, hair, carcasses, loose cells, and the like. Samples can be analyzed in the field utilizing modern techniques of DNA sequencing known as metagenomics, metabarcoding single species detection, and the like. Accurate detection in the field allows for rapid monitoring of biodiversity, environmental health, and extant population demographic analysis.

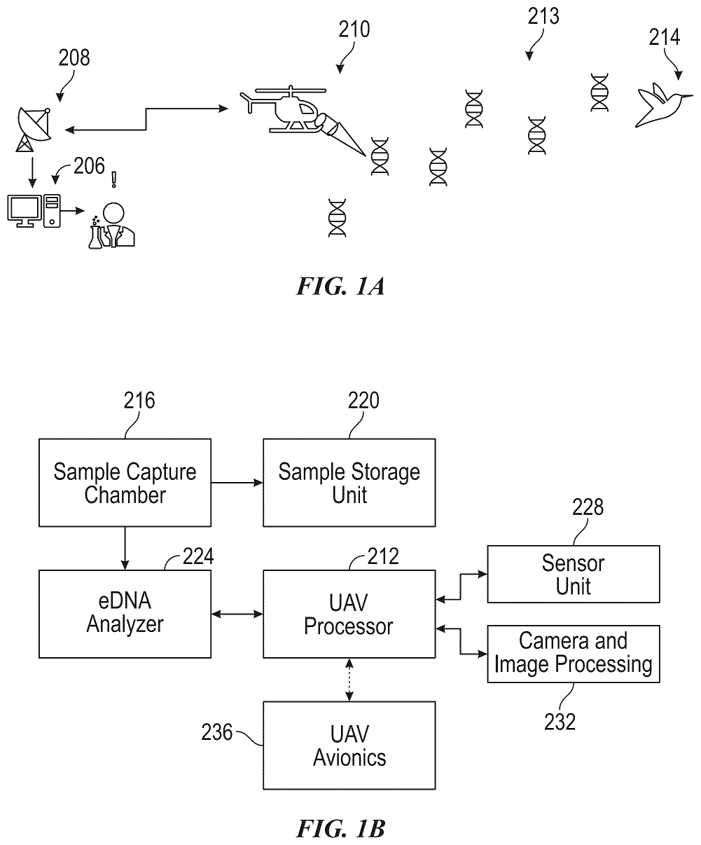

FIG. 1A is a high-level diagram of an eDNA UAV 210 configured to perform remote surveillance in an aerial environment, in accordance with an example embodiment. Equipping an unmanned aerial vehicle 210 with the capability to collect and evaluate eDNA 213 from a subject (e.g., bird 214) in the field enables novel conservation efforts to understand the genetic composition of regions that may be difficult or dangerous to reach. Moreover, the process can be automated with an autonomous UAV 210. In one example embodiment, techniques are provided to detect and evaluate eDNA 213 remotely via a UAV 210 for the purpose(s) of sampling extant populations, monitoring environmental biodiversity, facilitating conservational efforts, and the like, without the need to tranquilize and harvest samples from living organisms. Analysis results may be transmitted directly to a base station 206, via a repeater 208, and the like. Example embodiments provide the ability to study and monitor species that are invasive, elusive, or endangered without introducing anthropogenic stressors due to human interactions. Example embodiments enable the detection of pests in and around crops, soil, water, air, and the like; environmental surveillance and protection; and/or the detection of harmful pathogens in urban areas (such as measles). Given the teachings herein, the skilled person can adapt known UAVs capable of carrying payloads with an analysis system, such as known qPCR systems, to implement embodiments of the disclosed UAV system.

### UAV Environmental Surveillance

Unmanned aerial vehicles (UAVs) 210 can be remotely piloted, can be semi-autonomous (such as set to traverse a predetermined route, programmed to explore data at set geographical points, and the like), or can be fully autonomous. For example, image processing may be performed to identify a location (such as an area populated by ash trees) where samples are to be taken and then to only take samples around the ash trees to inspect for the presence of, for example, the invasive species Agrilus planipennis (Emerald Ash Borer)). In example embodiments, eDNA 213 sample collection and evaluation times are expedited, enabling faster, safer environmental surveillance and facilitating more comprehensive protection of species and biomes. In example embodiments, the surveillance of rare or endangered species is enabled and is used to study diverse populations without introducing stress to the organisms that could result in injury or death, and/or to surveil urban areas for deadly pathogens, drastically reducing response time and improving the success and viability of treatment plans.

eDNA UAV

In one example embodiment, an eDNA UAV 210 is configured for remote surveillance of, for example, dangerous and/or hard to reach locations. FIG. 1B is a high-level block diagram of an eDNA UAV 210 configured to perform remote surveillance of an environment, in accordance with an example embodiment. A sample capture chamber 216 on the UAV 210 facilitates the capture of DNA in a target environment. The eDNA 213 sampled may be captured while suspended in air by using the motion of the UAV 210, via manipulation of the location of the sample capture chamber 216 to collect the sample via an air intake valve, and the like. In one example embodiment, a purifying gas, such as ozone, is used to sanitize the sample capture chamber 216 between the collection of different samples.

It is noted that the performance of a qPCR analysis typically takes on the order of minutes. Therefore, in one example embodiment, the sample capture chamber 216 includes a plurality of sub-chambers to assist in pipelining the collection and analysis process. For example, a sample captured in a first sub-chamber of the sample capture chamber 216 may be analyzed while another sample is captured by a second sub-chamber of the sample capture chamber 216. FIG. 1C is a block diagram of an example sample capture chamber 216 that includes a plurality of sub-chambers 242-1, 242-2, in accordance with an example embodiment. In one example embodiment, a sample is collected through air intake 240 which is connected via an air-tight seal 244 to rotating sample capture chamber 216. When a sample is collected, air intake 240 closes and sample sub-chamber 242-1 rotates so that a clean/empty sub-chamber 242-2 is now connected to the air intake 240. A container 246 of fluorescent dyes/DNA probes/enzymes required for qPCR is connected to sample sub-chamber 242-1, either through the center of the rotating sample capture chamber 216, or in the sidewall of the UAV 210 so that it would only be able to release chemicals while the sample sub-chamber 242-1 is in a specific carousel position.

In one example embodiment, an eDNA analyzer 224 analyzes the samples captured in the sample capture chamber 216. The eDNA analyzer 224 may be, for example, a quantitative polymerase chain reaction (qPCR) analyzer. In one example embodiment, the eDNA analyzer 224 has onboard access to compounds, such as fluorescent dyes (such as an asymmetrical cyanine dye and the like) or DNA probes containing a fluorophore, such as hydrolysis probes), to measure the amount of amplified color product in real time and enable the amplification of specific fragments of the target species being searched for. The enzymatic chains may be temperature controlled to extend their shelf life on the UAV 210. In one example embodiment, the eDNA analyzer 224 is capable of performing an analysis on a plurality of samples in parallel, such as a plurality of samples residing in a plurality of sub-chambers in the sample capture chamber 216. In one example embodiment, the eDNA analyzer 224 is configured to pipeline the analysis of each sample such that a single analyzer may process a plurality of samples in parallel. The sample may also be stored in a sample storage chamber 220 for future analysis, either before, during, or after analysis. For example, a sample may be analyzed onboard and then stored for future further analysis. In one example embodiment, the samples stored within the sample storage chamber 220 is thermally-controlled to extend the usable life of the sample.

In one example embodiment, a UAV processor 212 is configured to control the capture of the sample by the sample capture chamber 216, the analysis of the sample by the eDNA analyzer 224, and the storage of the sample by the sample storage chamber 220. The UAV processor 212 interfaces with the UAV avionics 236 to, for example, issue commands to the UAV avionics 236 to relocate the UAV 210 to a new location, a new altitude, and the like. The UAV processor 212 interfaces with the sensor unit 228 to obtain information derived by sensors of the UAV 210 (such as to determine the location of the UAV 210 via, for example, a GPS receiver), and interfaces with the camera and image processing unit 232 to, for example, identify a target environment for collecting samples.

In one example embodiment, an UAV avionics unit 236 provides an interface for controlling the movement of the UAV 210 based on commands from the UAV processor 212. For example, the UAV processor 212 issues commands to the UAV avionics unit 236 to take-off, land, travel to a particular location or in a particular direction, and the like. The wireless capabilities of the UAV avionics unit 236 may also be used to provide seamless data transfer to external devices or cloud platforms for, for example, further analysis and storage of a report of the analysis results.

In one example embodiment, a camera and image processing unit 232 captures images from the UAV 210 and processes the captured images to, for example, identify a target environment. For example, the camera and image processing unit 232 may identify an ash tree in the pursuit of the invasive species Agrilus planipennis (Emerald Ash Borer). In one example embodiment, a sensor unit 228 includes various sensors, such as a GPS receiver for determining the location of the UAV 210.

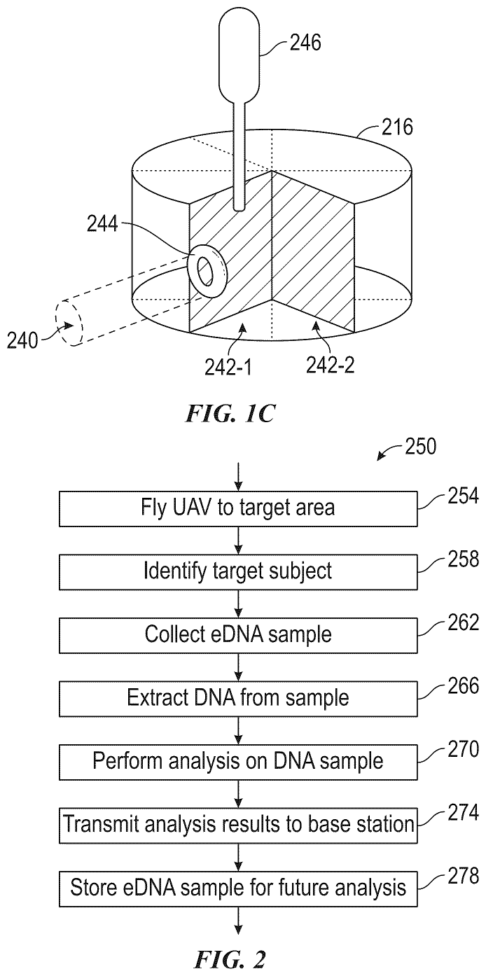

FIG. 2 is a flowchart 250 for controlling an eDNA UAV 210 configured to perform remote surveillance of an environment, in accordance with an example embodiment. Techniques that can be tailored to implement the steps of the flowchart 250 are well known in the corresponding arts. In one example embodiment, the UAV 210 is flown to a target area (operation 254). The target area may be specified by a user based, for example, on environmental maps, may be searched for by the UAV 210 given a target sampling entity, and the like. For example, the UAV 210 may use the on-board sensors 228 and/or the camera and image processing unit 232 to identify an area of interest, such as an area populated with a particular species of trees.

A target subject is identified (operation 258). The target subject may be a particular location for collecting the sample (such as a particular location identified by geographical coordinates and altitude), a particular object (such as a species of tree identified via the camera and image processing unit 232), and the like.

One or more samples are collected (operation 262). For example, samples may be collected by filtering air through the sample capture chamber 216 of the UAV 210 in-flight, by using a robotic arm to retrieve a sample from a surface of an object, and the like. In one example embodiment, the sample is tagged with the location of where and when the sample was collected. In one example embodiment, a purifying gas, such as ozone, is used to sanitize the sample capture chamber 216 between the collection of different samples.

DNA is extracted from the collected sample(s) and purified to remove chemicals, such as humic acid and the like, that would, for example, impair the analysis, such as inhibit a polymerase chain reaction (PCR) (operation 266). (It is noted that other variants of polymerase chain reaction are also contemplated.)

An analysis is performed on the extracted DNA sample, such as Mitochondrial DNA (mtDNA) (operation 270). In is noted that mtDNA has significant divergence across species and is represented thousands of times per cell, increasing viability of a technique with low levels of eDNA 213. In one example embodiment, qPCR is used to perform the analysis. In one example embodiment, the eDNA analyzer 224 is used to perform an analysis on a plurality of samples in parallel, such as a plurality of samples residing in a plurality of sub-chambers in the sample capture chamber 216.

The detection and identification of various organic materials, such as a pathogen, may also be performed. In one example embodiment, a target sequence is detected via a quantitative polymerase chain reaction (qPCR) creating billions of copies of a target sequence from the sampled eDNA 213 (which can be present in minute quantities). A sequence is then detected in real time via fluorescent signal amplification. If amplification occurs and the fluorescent signal is detected, the environmental sample is considered positive for the species of interest. In one example embodiment, only samples that are considered positive are stored for future analysis.

An analysis report is compiled and transmitted to, for example, a base station 206 (operation 274). In one example embodiment, analysis results are incorporated into a blockchain to facilitate environmental conservation, prevention of invasive species migration, healthcare, and the like. Blockchain may prove effective for protecting confidential information related to environmental samples, in ensuring that data is not being falsified, altered, or tampered with; in evaluating where the data (such sample data) is coming from and who has had access to the data, how data changes at each stage of the deployment of the UAV 210, and understanding the geotagging of the journey of samples and related data. Such capabilities are helpful for damage control, for example, in the case of a pathogen in an urban area. For invasive species, blockchain facilitates a better understanding of timelines, preserving the integrity of the data and geotagging.

The DNA sample is stored for future analysis (operation 278). For example, the DNA sample may be stored for retrieval and lab analysis when the UAV 210 lands. A wide-array of real-time data analysis algorithms for the interpretation of PCR results may be performed on the UAV 210 and transmitted to off-board systems and/or may be performed by off-board systems.

Benefits of Utilizing eDNA Detection

Example embodiments enable the collection and analysis of eDNA 213 without requiring observation and/or the capturing of a species of interest, and without requiring extensive documentation or human effort. Note that eDNA detection has also been shown to be more sensitive than traditional methods. These factors offer significant advantages in the early detection of invasive species and endangered species monitoring. The use of a qPCR-enabled UAV 210 enables the determination of prevalence of a species in an environment, and population statistics, without needing to count or capture individuals, and enables the study of public health via pathogen detection.

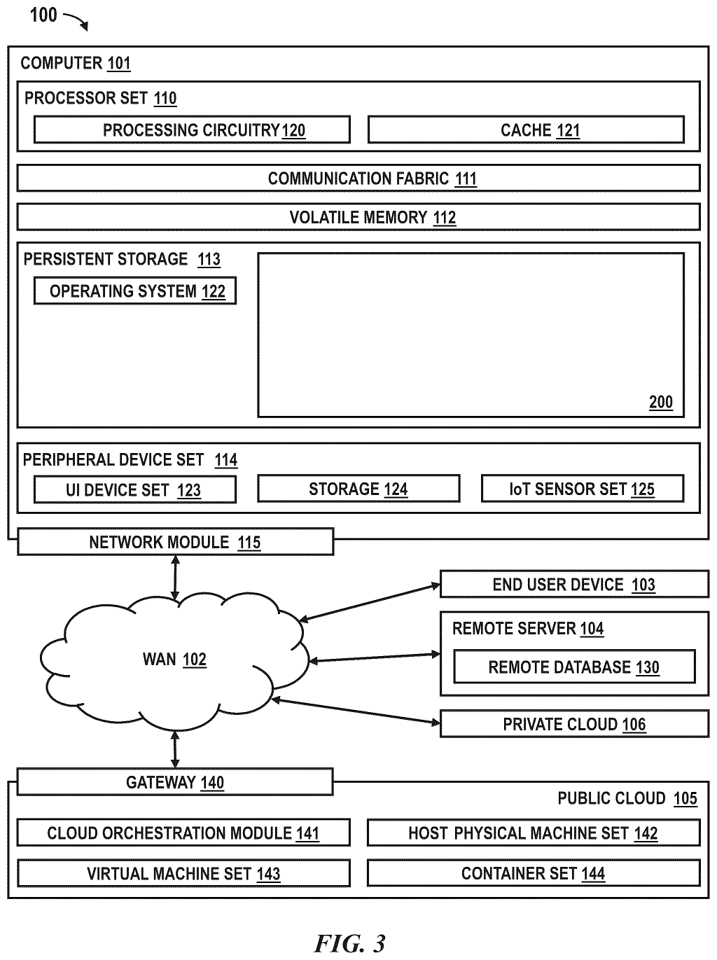

Refer now to FIG. 3.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as a UAV control system 200. In one example embodiment, the UAV processor 212 and/or the UAV avionics 236 execute some or all of the code in block 200 for control of a UAV system. On-board avionics may of course include one or more processors without necessarily having peripherals or network connections associated with a desktop or laptop computer. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 3. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An unmanned aerial vehicle comprising:
a sample capture chamber configured to collect a sample from a given location;
an environmental deoxyribonucleic acid (eDNA) analyzer configured to extract environmental deoxyribonucleic acid (eDNA) from the collected sample and to perform a quantitative polymerase chain reaction (qPCR) analysis on the extracted environmental deoxyribonucleic acid (eDNA); and
an unmanned aerial vehicle (UAV) processor configured to record results of the analysis, the results comprising an identification of the given location.

2. The apparatus of claim 1, further comprising a camera and image processing unit coupled to the unmanned aerial vehicle (UAV) processor and configured to capture and process an image to identify the given location.

3. The apparatus of claim 1, wherein the sample capture chamber further comprises an air intake valve configured to capture the sample from an atmosphere.

4. The apparatus of claim 1, wherein the sample capture chamber further comprises a purifying mechanism configured to pump ozone into the sample capture chamber to sanitize the sample capture chamber.

5. The apparatus of claim 1, wherein the sample capture chamber further comprises a plurality of sub-chambers configured to collect and analyze the collected sample and one or more additional samples in a pipelined manner.

6. The apparatus of claim 1, wherein the environmental deoxyribonucleic acid (eDNA) analyzer further comprises one or more enzyme containers, each enzyme container configured to hold a corresponding enzyme for replicating specific fragments of a given target species.

7. The apparatus of claim 1, wherein the environmental deoxyribonucleic acid (eDNA) analyzer is further configured to detect a target deoxyribonucleic acid (DNA) sequence based on a result of the quantitative polymerase chain reaction (qPCR) analysis.

8. The apparatus of claim 1, further comprising a sample storage chamber configured to store the sample in the sample capture chamber for future analysis.

9. The apparatus of claim 1, further comprising a sensor unit coupled to the unmanned aerial vehicle (UAV) processor and configured to determine a current location of the apparatus.

10. A method comprising:
collecting an environmental deoxyribonucleic acid (eDNA) sample on an unmanned aerial vehicle;
extracting deoxyribonucleic acid (DNA) from the collected environmental deoxyribonucleic acid (eDNA) sample on the unmanned aerial vehicle;
performing an analysis on the extracted environmental deoxyribonucleic acid (eDNA) on the unmanned aerial vehicle; and
transmitting a result of the analysis.

11. The method of claim 10, further comprising sanitizing, using ozone, a sample capture chamber following the collection of each environmental deoxyribonucleic acid (eDNA) sample.

12. The method of claim 10, further comprising purifying the extracted environmental deoxyribonucleic acid (DNA) on the unmanned aerial vehicle.

13. The method of claim 10, further comprising flying the unmanned aerial vehicle (UAV) to a target location.

14. The method of claim 13, further comprising capturing an image from the unmanned aerial vehicle, performing image processing on the captured image, and identifying the target location based on the image processing.

15. The method of claim 14, further comprising repeating the performance of the image processing and the identifying the target subject based on the image processing.

16. The method of claim 14, further comprising tagging the collected environmental deoxyribonucleic acid (eDNA) sample with one or more of a collection location and a time of collection.

17. A computer program product, comprising:
one or more tangible computer-readable storage media and program instructions stored on at least one of the one or more tangible computer-readable storage media, the program instructions executable by a processor, the program instructions comprising:
collecting an environmental deoxyribonucleic acid (eDNA) sample on an unmanned aerial vehicle;
extracting deoxyribonucleic acid (DNA) from the collected environmental deoxyribonucleic acid (eDNA) sample on the unmanned aerial vehicle;
performing an analysis on the extracted environmental deoxyribonucleic acid (eDNA) on the unmanned aerial vehicle; and
transmitting a result of the analysis.

18. The computer program product of claim 17, the program instructions further comprising sanitizing, using ozone, a sample capture chamber following the collection of each environmental deoxyribonucleic acid (eDNA) sample.

19. The computer program product of claim 17, the program instructions further comprising purifying the extracted deoxyribonucleic acid (DNA) on the unmanned aerial vehicle.

20. The computer program product of claim 19, the program instructions further comprising tagging the collected environmental deoxyribonucleic acid (eDNA) sample with one or more of a collection location and a time of collection.
